(19) United States
(12) Patent Application Publication
(10) Pub. No.: US 2024/0058411 A1
(43) Pub. Date: Feb. 22, 2024

(54) COMPOSITION FOR TREATING AND/OR PREVENTING A HANGOVER

(71) Applicant: Owen Garratt, Spruce Grove (CA)

(72) Inventor: Owen Garratt, Spruce Grove (CA)

(21) Appl. No.: 17/892,257

(22) Filed: Aug. 22, 2022

(51) Int. Cl. A61K 36/76; A61K 36/906; A61K 31/198; A61K 31/375; A61K 33/06; A61K 31/385; A61K 33/30; A61K 31/59; A61K 31/51; A61K 31/525; A61K 31/455; A61K 31/197; A61K 31/4188; A61K 31/714; A61K 33/04; A61K 31/145; A61K 33/00; A61K 31/12; A61P 25/32

(52) U.S. Cl. CPC A61K 36/76; A61K 36/906; A61K 31/198; A61K 31/375; A61K 33/06; A61K 31/385; A61K 33/30; A61K 31/59; A61K 31/51; A61K 31/525; A61K 31/455; A61K 31/197; A61K 31/4188; A61K 31/714; A61K 33/04; A61K 31/145; A61K 33/00; A61K 31/12; A61P 25/32

(57) ABSTRACT

The invention is directed to a composition for treating and/or curing a hangover. Specifically, the disclosed composition targets the unpleasant physiological effects that occur as a result of drinking alcohol beyond the drinker's metabolic capability. Advantageously, the disclosed composition targets the various different symptoms of alcohol consumption, and also provides nutritional support for the metabolization of alcohol. The disclosed formulation of vitamins, minerals, electrolytes, amino acids, salicin, and gingerols provide an effective treatment to eliminate and/or treat the effects of the hangover, while also promoting the metabolization of alcohol.

### TECHNICAL FIELD

The presently disclosed subject matter is generally directed to a composition for treating and/or preventing a hangover in a subject.

### BACKGROUND

A hangover is described as a group of unpleasant symptoms that often develop after drinking too much alcohol. The term “alcohol” typically refers to ethyl alcohol (ethanol), which is the common form of consumable alcohol found in beer, wine, and liquor. After consumption, the alcohol passes through the stomach and into the bloodstream, where it is distributed throughout the body, irritating and damaging cells and cell membranes. As the body metabolizes the alcohol, acetaldehyde is created in the liver. The acetaldehyde is automatically attacked by acetaldehyde dehydrogenase and glutathione. The process works well, leaving the acetaldehyde only a short time to adversely affect the body. However, the amount of glutathione stored in the liver is quickly depleted when large amount of alcohol is consumed. As a result, the acetaldehyde builds up in the body, causing a hangover. The symptoms of a hangover can include nausea, vomiting, headache, muscle ache, dry mouth, fatigue, weakness, shakiness, decreased ability to concentrate, reduced sleep quality or duration, mood disturbances, thirst, and/or rapid heartbeat. Alcohol hangovers vary greatly in severity, which correlates strongly with degree of alcohol intoxication preceding the alcohol hangover. Genetic variation can also affect alcohol hangover severity.

Hangovers typically occur after most, if not all, of the alcohol is cleared from a drinker's body. The drinker may have slept for several hours as well. Accordingly, a hangover generally does not occur during acute alcohol intoxication. Clearance of alcohol from a drinker's body is typically achieved through metabolism of alcohol to acetaldehyde, which is subsequently metabolized to acetic acid, and then to carbon dioxide. Some alcohol is also excreted unchanged. The hangover symptoms often arise as one's blood alcohol level returns to normal levels after an episode of drinking. For example, if an episode of drinking occurs in the evening, the hangover symptoms typically appear the following morning.

Many hangover remedies are widely available, although most are relatively ineffective and/or include serious drawbacks. For instance, treatment of a hangover with acetaminophen (which can be effective at reducing headache) can cause liver damage or exacerbate liver damage caused by alcohol or acetaldehyde. Users also frequently drink large volumes of fluid, such as coffee, water, sports drinks in an effort to prevent or treat a hangover. However, these methods are relatively ineffective at mitigating hangover symptoms. Various types of pills are also marketed as targeted cures for a hangover, although these fail to treat the variety of symptoms present. For example, a pill to treat a headache lacks the ability to combat nausea.

It would therefore be advantageous to provide a composition and method for treating a hangover that reliably eliminates or improves hangover symptoms.

### SUMMARY

In some embodiments, the presently disclosed subject matter is directed a composition for treating, preventing, or both treating and preventing a hangover. Specifically, a single dose of the composition comprises white willow bark extract in an amount of about 300 mg, ginger extract in an amount of about 20 mg, glutamine in an amount of about 500 mg, vitamin C in an amount of about 500 mg, taurine in an amount of about 100 mg, magnesium citrate in an amount of about 100 mg, potassium gluconate in an amount of about 100 mg, alpha lipoic acid in an amount of about 50 mg, zinc picolinate in an amount of about 10 mg, vitamin D in an amount of about 500 IU, vitamin B1 in an amount of about 25 mg, vitamin B2 in an amount of about 25 mg, vitamin B3 in an amount of about 25 mg, vitamin B5 in an amount of about 8 mg, vitamin B7 in an amount of about 0.4 mg, vitamin B12 in an amount of about 0.1 mg; and selenium in an amount of about 0.1 mg.

In some embodiments, the composition is an oral supplement in the form of a capsule, tablet, or powder.

In some embodiments, the white willow bark comprises about 15 weight percent salicin, based on the total weight of the white willow bark included in the composition.

In some embodiments, the ginger root extract comprises about 20 weight percent gingerol, based on the total weight of the ginger root extract.

In some embodiments, the components are combined to generate a powder form or liquid form compound taken prior to, during, or after consumption of an alcoholic beverage.

In some embodiments, the composition further includes one or more additives present in an amount of 5 weight percent or less of the total weight of the composition.

In some embodiments, the presently disclosed subject matter is directed to a method of treating, preventing, or both treating and preventing a hangover in a subject, the method comprising administering the disclosed composition to a subject.

In some embodiments, the subject has a blood alcohol level of at least 0.01.

In some embodiments, the administering occurs while the subject is drinking alcohol.

In some embodiments, the administering occurs prior to onset of symptoms of a hangover in the subject.

In some embodiments, the administering occurs after the onset of symptoms of a hangover in the subject.

In some embodiments, administering comprises orally administering one, two, or three doses per day to the subject.

In some embodiments, the presently disclosed subject matter is directed to a kit comprising the disclosed composition in one or more unit dose forms.

In some embodiments, the composition of the kit is administered to the subject prior to onset of symptoms of a hangover.

In some embodiments, the composition of the kit is administered to the subject after the onset of symptoms of a hangover.

In some embodiments, the composition of the kit is administered while the subject is drinking alcohol.

### DETAILED DESCRIPTION

The presently disclosed subject matter is introduced with sufficient details to provide an understanding of one or more particular embodiments of broader inventive subject matters. The descriptions expound upon and exemplify features of those embodiments without limiting the inventive subject matters to the explicitly described embodiments and features. Considerations in view of these descriptions will likely give rise to additional and similar embodiments and features without departing from the scope of the presently disclosed subject matter.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in the subject specification, including the claims. Thus, for example, reference to “a device” can include a plurality of such devices, and so forth. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise indicated, all numbers expressing quantities of components, conditions, and so forth used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, and/or percentage can encompass variations of, in some embodiments+/−20%, in some embodiments+/−10%, in some embodiments+/−5%, in some embodiments+1-1%, in some embodiments+/−0.5%, and in some embodiments +/−0.1%, from the specified amount, as such variations are appropriate in the disclosed packages and methods.

As used herein, the term “and/or” includes any and all combinations of one or more of the associated listed items.

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

The presently disclosed subject matter is directed to a composition for treating and/or curing a hangover. The term “hangover” refers to the experience of unpleasant physiological effects that occur as a result of drinking alcohol beyond the drinker's metabolic capability. As noted above, alcohol (ethanol) is metabolized to acetaldehyde, which can remain at a high plateau level in the body after the initial consumption of ethanol, resulting in a hangover. Advantageously, the disclosed composition targets the various different symptoms of alcohol consumption, and also provides nutritional support for the metabolization of alcohol. The disclosed formulation of vitamins, minerals, electrolytes, amino acids, salicin, and gingerols provide an effective treatment to eliminate and/or treat the effects of the hangover, while also promoting the metabolization of alcohol.

The disclosed composition incudes salicin to treat headaches, ginger extract to treat nausea, electrolytes for rehydration, vitamin C for antioxidant protection of the liver, magnesium to promote proper sleep and other advantageous processes, and amino acids, minerals, and nutrients to aid with processing of alcohol metabolites (e.g., acetaldehyde and acetate). One example of the disclosed composition (a single dose) is set forth in Table 1 below:

TABLE 1

| | | |
|---|---|---|
| Component | Amount/Dose | Wt % |

| | | | |
|---|---|---|---|
| White Willow Bark Extract | 300 | mg | 17.01 |
| (Standardized to 15% | | | |
| Salicin) | | | |
| Ginger Extract (20% | 20 | mg | 1.13 |
| ginerols) | | | |
| Glutamine | 500 | mg | 28.35 |
| Vitamin C | 500 | mg | 28.35 |
| Taurine | 100 | mg | 5.67 |
| Magnesium Citrate | 100 | mg | 5.67 |
| Potassium Gluconate | 100 | mg | 5.67 |
| Alpha Lipoic Acid | 50 | mg | 2.84 |
| Zinc Picolinate | 10 | mg | 0.57 |

| | | |
|---|---|---|
| Vitamin D | 500 IU | 0.00071 |
| | (0.0125 mg) | |

| | | | |
|---|---|---|---|
| B1 Thiamin | 25 | mg | 1.42 |
| B2 Riboflavin | 25 | mg | 1.42 |
| B3 Niacin | 25 | mg | 1.42 |
| B5 Pantothenic Acid | 8 | mg | 0.45 |
| B7 Biotin | 0.4 | mg | 0.225 |
| B12 Methylcarbylamine | 0.1 | mg | 0.0057 |
| Selenium | 0.1 | mg | 0.0057 |

Thus, the disclosed composition includes salicin in an effective amount (e.g., about 300 mg). The term “salicin” refers to 2-hydroxymethylphenyl β-D-glucopyranoside or saligenin-β-glucoside. Salicin is a compound found in the willow bark or white willow bark plant. The extract of willow bark contains salicinium which reduces headache pain. The pain-relieving effects of salicin are similar to those of aspirin without side effects, such as stomach upset. Salicin is converted into aspirin in vivo and provides a delayed pain relieving affect after the ingestion of the compositions disclosed herein. Therefore, stomach upset is delayed or may not be perceived due to the pain relieving effects of the alcohol ingested. Although not bound by any theory, it is believed that the inhibitory effect of salicin on prostaglandin synthesis in nerve cells is the mechanism by which salicin relieves pain. White willow bark extract and/or pure salicin in the concentration range of about 300 mg/dose is used in the disclosed compositions to provide relief from hangover symptoms. Thus, the composition can include salicin in an amount of from about 200-400 mg per dose (e.g., at least/no more than about 200 mg, 250 mg, 300 mg, 350 mg, or 400 mg per dose). The unit dosage is the amount of the composition formulated for a single dose/serving. Thus, the term “per dose” refers to the recommended dosage and can include a capsule, tablet, or other unit amount taken by a subject at a particular time to treat a condition (e.g., achieve the desired therapeutic effect).

The white willow bark comprises about 15 weight percent salicin, based on the total weight of the white willow bark. Thus, the white willow bark can include at least about (or no more than about) 10, 15, or 20 weight percent salicin.

The disclosed composition further includes ginger extract as an anti-emetic to treat nausea and/or vomiting. Ginger root extract is derived from the root of the herb Zingiber officionale which grows widely in southwest India. Ginger is a popular spice in Indian cooking, and its medicinal uses have been well documented. As such, ginger root offers many benefits. Historically, ginger root has been used to ease menstrual cramps, treat seasickness and food poisoning, and to eliminate body odor. It has also been determined that ginger root can be used to treat nausea and vomiting associated with hangovers. The disclosed composition can include about 20 mg of the ginger extract per dose (e.g., at least/no more than about 10, 15, 20, 25, or 30 mg ginger extract).

The ginger root extract can include about 20 weight percent gingerols, based on the total weight of the ginger root extract. The term “gingerol” refers to 6-gingerol, a phenol phytochemical compound found in ginger root extract. In some embodiments, the term “gingerol” includes 8-gingerol, 10-gingerol, and 12-gingerol.

The disclosed composition also includes antioxidants to provide beneficial effects for the user. For example, vitamin C and alpha-lipoic acid can be included. Specifically, vitamin C (also known as ascorbic acid and ascorbate) is a water-soluble vitamin that acts as an essential nutrient involved in the repair of tissue, such as protection of the liver. Thus, vitamin C is involved in all phases of wound healing. For example, in the inflammatory phase, it is required for neutrophil apoptosis and clearance. During the proliferative phase, vitamin C contributes towards synthesis, maturation, secretion, and degradation of collagen. Vitamin C also acts as a strong antioxidant, relieving oxidative stress by preventing the formation and oxidation of free radicals. In addition, vitamin C supports a healthy immune system. Vitamin C also helps speed up the metabolism of alcohol in the liver, which helps alleviate hangover symptoms sooner. The disclosed composition comprises about 500 mg vitamin C per dose (e.g., at least/no more than about 400, 450, 500, 550, or 600 mg).

The composition further includes the antioxidant alpha-lipoic acid that has been shown to be useful in aerobic metabolism. Alpha-lipoic acid is an organosulfur compound derived from caprylic acid (octanoic acid) and is a naturally occurring antioxidant. Specifically, alpha-lipoic acid helps produce glutathione, one of the most powerful antioxidants and a crucial substance for many processes in the body. Alpha-lipoic acid further helps recycle energy from other antioxidants and replenishes vitamin C and vitamin E levels in the body. Further, alpha-lipoic acid helps treat hangover-related oxidative stress and blood toxicity to help the body maintain balance. The disclosed composition comprises about 50 mg alpha-lipoic acid per dose (e.g., at least/no more than about 40, 45, 50, 55, or 60 mg).

Amino acids are also provided in the disclosed composition. For example, glutamine is an alpha amino acid used in the biosynthesis of proteins. The consumption of alcohol depletes the body of glutamine, creating an imbalance. As a result, the body produces glutamine which wreaks havoc on the sleep schedule, aggravates the stomach, and hinders the recovery process. Thus, by providing the body with glutamine after drinking alcohol, the imbalance and negative effects associated therewith are avoided. The disclosed composition can include about 500 mg/dose of glutamine (e.g., at least/no more than about 400, 450, 500, 550, or 600 mg/dose).

The amino acid taurine is also included in the disclosed composition and is believed to functionally increase the total activity of alcohol dehydrogenase (ALDH2). Importantly, ALDH2 catalyzes the chemical breakdown of acetaldehyde into acetic acid, which is less harmful to the body and can be excreted. As mentioned above, acetaldehyde is commonly formed form the breakdown of ethanol and is the byproduct associated with the symptoms of hangovers and skin flushing following excessive alcohol consumption. The disclosed composition can include about 100 mg taurine/dose (e.g., at least/no more than about 75, 80, 85, 90, 95, 100, 105, 110, 115, 120, or 125 mg/dose).

The composition can also include several B vitamins. Specifically, vitamin B1 (thiamine), vitamin B2 (riboflavin), vitamin B3 (niacin), vitamin B5 (Pantothenic aid), vitamin B7 (biotin), and vitamin B12 (methylcarbylamine) are included. The noted B vitamins are believed to combat the depressive effects of ethanol consumption, particularly vitamin B12. B vitamins are lost during the consumption of ethanol due to the diuretic effects of consuming alcohol. The B vitamins also boost energy levels, cell metabolism, brain functions, and the like. Thus, B vitamins act as antioxidants and coenzymes that service important roles in energy metabolism, nervous system health, digestive health, decreasing nausea, decreasing vomiting, facilitating concentration, and proper functioning of the immune system.

Specifically, vitamin B1 (thiamine) is generally known to support nerve and brain health, to promote gut health, to increase energy, and to combat the symptoms of stress. Vitamin B1 is required to form adenosine triphosphate, which serves as the source of energy for all cells. A deficiency of vitamin B1 can include nausea, headache, fatigue, abdominal discomfort, depression, and irritability.

Vitamin B2 (riboflavin) promotes energy production, serves as an antioxidant, and plays a role in red blood cell production. Symptoms of vitamin B2 deficiency can include digestive problems, fatigue, sensitivity to light, eye fatigue, and cracks and sores at the corners of the mouth. The two biologically active forms are flavin mono nucleotide (FMN) and flavin adenine dinucleotide (FAD) formed by the transfer of an AMP moiety from ATP to FMN. FMN and FAD are each capable of reversibly accepting two hydrogen atoms, forming FMNH2 or FADh2. FMN and FAD are bound tightly—sometimes covalently—to flavoenzymes that catalyze the oxidation or reduction of a substrate.

Vitamin B3 (niacin) helps promote energy production, serves as an antioxidant, improves circulation, and suppresses inflammation. Symptoms of Vitamin B3 deficiency can include fatigue, vomiting, depression, poor circulation, and indigestion. In more severe cases, Vitamin B3 can cause a constellation of symptoms including diarrhea, dementia, and cracked scaly skin, which is known as pellagra.

Vitamin B5 (pantothenic acid from Calcium D-Pantothenate) plays a role in carbohydrate and fat metabolism, red blood cell production, and the production of sex and stress hormones. It also plays a role in maintaining digestive tract health. Deficiencies of Vitamin B5 can lead to symptoms such as fatigue, vomiting, depression, irritability, stomach pain, insomnia, and upper respiratory infections.

Vitamin B7 (biotin) helps regulate blood sugar and plays a role in maintenance of the nervous system and key metabolic processes. Deficiencies can cause fatigue, muscle pain, dermatitis, dry skin, and intestinal dysfunction. Biotin is a coenzyme in carboxylation reactions, in which it serves as a carrier of activated carbon dioxide. Biotin is covalently bound to the epsilon-amino groups of lysine residues of biotin dependent enzymes. Biotin deficiency does not occur naturally because the vitamin is widely distributed in food.

Vitamin B12 (Cyanocobalamin) is required in humans for two essential enzymatic reactions: the synthesis of methionine and the isomerization of methylmalonyl CoA that arises from fatty acids with odd numbers of carbon atoms. When the vitamin is deficient, abnormal fatty acids accumulate and become incorporated into the cell membranes, including those of the nervous system, which can account for some of the neurologic manifestations of vitamin B12 deficiency.

Vitamins B1, B2, and B3 can be present in the disclosed composition each in an amount of about 25 mg/dose. Thus, the disclosed composition can include vitamins B1, B2, and B3 in an amount of at least about (or no more than about) 15, 20, 25, 30, or 35 mg/dose. Vitamin B5 can be present in the disclosed composition in an amount of about 8 mg/dose (e.g., at least/no more than about 5, 6, 7, 8, 9, 10, or 11 mg/dose). Vitamin B7 can be present in an amount of about 0.4 mg (e.g., at least/no more than about 0.2, 0.3, 0.4, 0.5, 0.6, 0.7, or 0.8 mg/dose). Vitamin B12 can be present in an amount of about 0.1 mg/dose (e.g., at least/no more than about 0.05, 0.1, 0.15 mg).

Several minerals and nutrients are also included in the disclosed composition to aid in processing the alcohol metabolites. The minerals and nutrients can help replenish nutrients that are lost or broken down due to alcohol consumption. For instance, where alcohol breaks down nutrients in the body or causes them to be excreted, the consumption of alcohol can cause nutrient deficiencies that lead to hangover symptoms (e.g., nausea, sensitivity to light and noise, etc.). Accordingly, minerals and nutrients can help to replenish the drinker's nutrient levels to reduce hangover symptoms associated with the alcohol-related nutrient deficiencies. Thus, the disclosed composition can include vitamin D, selenium, zinc picolinate, potassium gluconate, magnesium citrate. Vitamin D is a group of fat-soluble secosteroids responsible for increasing intestinal absorption of calcium, magnesium, and phosphate, among other biological effects. Advantageously, vitamin D also boosts the immune system, keeping it functioning normally. The disclosed composition includes about 500 IU of vitamin D per dose (e.g., at least/no more than about 400, 450, 500, 550, or 600 IU/dose).

Zinc picolinate is believed to aid in glucose tolerance by producing GTF (glucose tolerance factor). As such, zinc picolinate is believed to minimize the ability of alcohol to adversely affect glucose/insulin tolerance. In addition, zinc is a cofactor with selenium, glutathione, and other agents for alcohol metabolism. Zinc deficiency can lead to alcoholism and impaired alcohol induced glucose tolerance. The disclosed composition can include zinc picolinate in an amount of about 10 mg/dose. Thus, the composition can include at least about (or no more than about) 5, 6, 7, 8, 9, 10, 11, 12, 13, 14, 1 or 15 mg/dose.

Magnesium citrate is a magnesium preparation in salt form with citric acid in a 1:1 ratio (1 magnesium atom per citrate molecule). Magnesium citrate helps to relax neck and head muscles, thereby alleviating hangover tensions. Magnesium further prevents the thickening of blood in the vessels due to alcohol consumption, which could lead to clots. During a hangover, magnesium is used up by metabolically active immune cells and during the metabolism of alcohol. Magnesium citrate is believed to support the alcohol dehydrogenase enzyme that breaks down alcohol and eliminates it from the body. The disclosed composition comprises about 100 mg/dose of magnesium citrate (e.g., at least/no more than about 75, 80, 85, 90, 95, 100, 105, 110, 115, 120, 125 mg/dose).

Potassium gluconate is the potassium salt of the conjugate base of gluconic acid and is used as a mineral supplement and sequestrant. Alcohol acts as a diluent, and levels of potassium can be lost during drinking. Potassium gluconate can be used to treat low amounts of potassium in the blood. Potassium is an essential mineral, necessary for the heart and muscles to contract normally. Low levels of potassium can lead to sore muscles, weakness, and dizziness that are often noticed during a hangover. The disclosed composition includes about 100 mg/dose of potassium gluconate (e.g., at least/no more than about 75, 80, 85, 90, 95, 100, 105, 110, 115, 120, or 125 mg/dose).

Selenium is involved in the synthesis of the enzyme glutathione peroxidase (GP). GP consists of four subunit enzymes with one selenium per subunit in the form of selenocysteine. This enzyme is responsible for alcohol detoxification in the body. By combining selenium and glutathione in the proper ratios, alcohol breakdown in the human body is facilitated without excess production of aldehydes and other toxic metabolites in the bloodstream. These toxic metabolites are a major source of hangover symptoms. The disclosed composition can include about 0.1 mg/dose of selenium (e.g., at least/no more than about 0.05, 0.075, 0.1, 0.15, or 0.2 mg/dose of selenium).

The disclosed composition can optionally include one or more additives, such as (but not limited to) carriers, binders, filling agents, suspending agents, disintegrating agent, dispersing agent, surfactant, lubricant, diluent, solubilizer, stabilizer, flavoring agents, sweeteners, colorants, preservatives, and the like. The additives are well known in the art and can be present in an amount of about 5 weight percent or less of the composition.

The disclosed composition can have any suitable form that allows it to be orally ingested, consumed, or administered. Thus, the composition can be configured as a beverage, a tablet, a capsule, a pill, a chewable solid formulation, a food additive, a suspension, an elixir, a syrup, an emulsion, a powdered drink mix, and/or any other suitable form. For example, using standard coating procedures, such as those described in Remington's Pharmaceutical Sciences, 20th Edition (2000) (incorporated by reference herein), a film coating can be provided around the compositions.

In some embodiments, the presently disclosed subject matter includes a kit comprising the disclosed composition in one or more unit dose forms. For example, the kit can include one or more unit dose forms in a single serving drink formulation. In other embodiments, the kit can include one or more unit dose forms in a dry formulation, such as a powder or pill form. In another embodiments, the kit can include one or more tablets. In some embodiments, the kit can include packaging (e.g., a bottle, blister package, cylinder of powder, etc.) and/or directions for use. In this way, the kit can treat a single hangover with multiple doses and/or can treat multiple hangovers that occur on different occasions.

The disclosed composition can be used to prevent and/or treat a hangover, comprising the step of administering the composition to a subject having the symptoms of hangover or are at risk of having the symptoms of hangover. The term “preventing” refers to the prophylactic administration of the disclosed composition to substantially diminish the likelihood or severity of a hangover or to delay the onset of a hangover. As used herein, “treating” refers to a method of ameliorating a hangover or one or more of the biological manifestations of a hangover. In some embodiments, the term includes directly or indirectly interfering with one or more points in the biological cascade that leads to or is responsible for the hangover; or one or more of the biological manifestations of the hangover; to alleviate one or more of the symptoms, effects or side effects associated with the hangover; or to slow the progression of the hangover or one or more of the biological manifestations of the hangover.

In some embodiments, the composition can be administered as a single dose (e.g., before consuming alcohol, during the consumption of alcohol, and/or after the consumption of alcohol). In other embodiments, the composition is administered in multiple doses (e.g., 2 or more).

In some embodiments, administering comprises dissolving the composition in the form of a powder, tablet, or other solid in liquid and orally administering the composition and liquid mixture to a subject. In other embodiments, administering includes administering a capsule, tablet, or other solid element comprising the composition to a subject, which the subject then swallows. In other arrangements, administering comprises orally administering the composition in the form of a gel to a subject. The composition can also be configured as a food supplement that is added to a food item and then consumed.

The described composition can be used to reduce hangover symptoms in any suitable manner. In one example, the composition can be taken before the consumption of alcohol. Alternatively, the composition can be taken with alcohol. In still other embodiments, the composition can be taken at some period of time after the consumption of alcohol (e.g., as a chaser). In yet another example, the composition can be taken as needed. For instance, a person can administer the composition before, during and/or after the consumption of alcohol. Indeed, the specific dose level (e.g., the amount of ingredients in the composition and the amount of the composition that a person ingests) and the frequency of consumption for any particular person can be varied and will depend upon a variety of factors, including the age, body weight, general health, sex, diet, mode and time of administration, severity of the hangover, and so forth.

Examples of the hangover symptom(s) include, but are not limited to, fatigue, weakness, excessive thirst, dry mouth, bad breath, headache, muscle ache, nausea, vomiting, stomach pain, poor sleep, decreased sleep, increased light sensitivity, increased sound sensitivity, dizziness, sense of room spinning, shakiness, decreased ability to concentrate, mood disturbance, depression, anxiety, irritability, and rapid heartbeat, and combination thereof. In some embodiments, the hangover symptom(s) occur following excess consumption of alcoholic beverages that result in a blood alcohol level of 0.06% or greater (e.g., at least/no more than about 0.06, 0.08, 0.1, 0.15, 0.2, 0.25, 0.3, 0.35, 0.4, 0.45, 0.5, 0.55 or 0.6%).

As discussed above, the disclosed composition can have many beneficial characteristics. For example, the composition to reduce headache and muscle pain and other hangover symptoms while improving liver function. The composition can further replenish nutrients in the body that are lost or broken down as a result of alcohol consumption. By replenishing these nutrients, the composition helps the body return to its normal state of functioning and acts to reduce hangover symptoms. In some embodiments, the composition further acts to detoxify the body by removing harmful chemicals associated with alcohol. Additionally, because the composition is easily ingested, use of the composition can be convenient, especially where the composition has a pleasing taste. In another example of a beneficial characteristic, where the composition is caffeine free (e.g., comprises decaffeinated green tea extract), the composition, unlike some conventional chasers that comprise caffeine, may not impede sleep after being consumed. Furthermore, the described composition is not damaging to the stomach or liver, even when taken with alcohol.

The term “subject” can be a human in some embodiments. However, the presently disclosed subject matter is not limited and the disclosed methods can be used on any subject in need of prevention and/or treatment of a hangover. Thus, a subject can include farm animals (such as cows, sheep, and goats), sport animals, pets (such as cats, dogs, and horses), primates (such as, monkeys, gorillas, and chimpanzees), mice and rats.

Exemplary embodiments of the methods and components of the presently disclosed subject matter have been described herein. As noted elsewhere, these embodiments have been described for illustrative purposes only, and are not limiting. Other embodiments are possible and are covered by the presently disclosed subject matter. Such embodiments will be apparent to persons skilled in the relevant art(s) based on the teachings contained herein. Thus, the breadth and scope of the present invention should not be limited by any of the above-described exemplary embodiments but should be defined only in accordance with the following claims and their equivalents.

The features and advantages of the present invention are more fully shown by the following examples which are provided for purposes of illustration and are not to be construed as limiting the invention in any way.

### EXAMPLE

Testing of Subjects After Consuming Alcoholic Beverages subjects were administered one dose of the disclosed formulation at the cessation of drinking alcohol with 8-12 ounces of water. Prior to being administered the dose, each subject had consumed 2 or more alcoholic beverages per hour for at least 2 hours and were considered intoxicated. The subjects included both men and women of varying weight and age ranges. The dosage administered was that set forth in Table 1 above. After the dosage was administered, each subject went to sleep for the night and slept 6 or more hours.

Upon waking 100% of the subjects reported a reduction in hangover symptoms. Specifically, 90% of the subjects (18 out of 20) reported a complete absence of hangover symptoms after consuming a minimum of 5 alcoholic beverages.

5% of the subjects (1 out of 20) reported a 75% reduction in symptoms, and 5% reported a two-thirds reduction in symptoms. These two testers also consumed nearly twice the alcohol as the rest of the subjects. These two testers were given a second dose upon waking and reported their remaining symptoms had disappeared within 30 minutes.

All testers reported they had slept better, had no headache or nausea, and had no anxiousness or feelings of dread compared to how they would typically expect to feel after consuming the same amount of alcohol.

What is claimed is:

1. A composition for treating, preventing, or both treating and preventing a hangover, a single dose of the composition comprising:
white willow bark extract in an amount of about 300 mg;
ginger extract in an amount of about 20 mg;
glutamine in an amount of about 500 mg;
vitamin C in an amount of about 500 mg;
taurine in an amount of about 100 mg;
magnesium citrate in an amount of about 100 mg;
potassium gluconate in an amount of about 100 mg;
alpha lipoic acid in an amount of about 50 mg;
zinc picolinate in an amount of about 10 mg;
vitamin D in an amount of about 500 IU;
vitamin B1 in an amount of about 25 mg;
vitamin B2 in an amount of about 25 mg;
vitamin B3 in an amount of about 25 mg;
vitamin B5 in an amount of about 8 mg;
vitamin B7 in an amount of about 0.4 mg;
vitamin B12 in an amount of about 0.1 mg; and
selenium in an amount of about 0.1 mg.

2. The composition of claim 1, wherein the composition is an oral supplement in the form of a capsule, tablet, or powder.

3. The composition of claim 1, wherein the white willow bark comprises about 15 weight percent salicin, based on the total weight of the white willow bark included in the composition.

4. The composition of claim 1, wherein the ginger root extract comprises about 20 weight percent gingerol, based on the total weight of the ginger root extract.

5. The composition of claim 1, wherein the components are combined to generate a powder form or liquid form compound taken prior to, during, or after consumption of an alcoholic beverage.

6. The composition of claim 1, further comprising one or more additives present in an amount of 5 weight percent or less of the total weight of the composition.

7. A method of treating, preventing, or both treating and preventing a hangover in a subject, the method comprising administering the composition of claim 1 to a subject.

8. The method of claim 7, wherein the subject has a blood alcohol level of at least 0.01.

9. The method of claim 7, wherein the administering occurs while the subject drinking alcohol.

10. The method of claim 7, wherein the administering occurs prior to onset of symptoms of a hangover in the subject.

11. The method of claim 7, wherein administering occurs after the onset of symptoms of a hangover in the subject.

12. The method of claim 7, wherein administering comprises orally administering one, two, or three doses per day to the subject.

13. The method of claim 7, wherein the white willow bark comprises about 15 weight percent salicin, based on the total weight of the white willow bark included in the composition.

14. The method of claim 7, The composition of claim 1, wherein the ginger root extract comprises about 20 weight percent gingerol, based on the total weight of the ginger root extract.

15. A kit comprising the composition of claim 1 in one or more unit dose forms.

16. The kit of claim 15, wherein the composition of the kit is administered to a subject prior to onset of symptoms of a hangover.

17. The kit of claim 15, wherein the composition of the kit is administered to a subject after the onset of symptoms of a hangover.

18. The kit of claim 15, wherein the composition of the kit is administered to a subject while the subject is drinking alcohol.

19. The kit of claim 15, wherein the white willow bark comprises about 15 weight percent salicin, based on the total weight of the white willow bark included in the composition

20. The kit of claim 15, wherein the ginger root extract comprises about 20 weight percent gingerol, based on the total weight of the ginger root extract.
